(19) United States
(12) Patent Application Publication
(10) Pub. No.: US 2008/0086327 A1
(43) Pub. Date: Apr. 10, 2008

(54) System and method for determining and verifying disease classification codes

(75) Inventors: Michael Cox, Point Pleasant, NJ (US); David Leeney, East Hampton, NY (US); Eric Pedrotty, Neptune, NJ (US); John Citrodella, Brick, NJ (US); Narinder Bhalta, Roanoka, VA (US)

Correspondence Address: LAW OFFICES OF JOHN R. MUGNO, 350 BROADWAY - 10TH FLOOR, NEW YORK, NY, 10013, US

(73) Assignee: QMED, INC.

(21) Appl. No.: 11/544,804

(22) Filed: Oct. 6, 2006

(51) Int. Cl. G06Q 10/00; A61B 5/00

(52) U.S. Cl. 705/2; 600/300

(57) ABSTRACT

A system and method for determining an updated disease classification code for a patient within a managed care population consisting of (i) a patient condition processing unit for receiving a plurality of patient-related data, (ii) a diagnosis repository database coupled to the patient condition processing unit for storing a preestablished disease classification code for the patient, and (iii) a disease classification code application tool designed to convert medical chart data of the patient into an observed disease classification code for the patient wherein the observed disease classification code is forwarded to the patient condition processing unit and stored in a diagnosis repository database as the updated disease classification code. The updated disease classification code can then be forwarded to the treating physician, reimbursement agency, or any other agency requiring such data. The patient-related data can consist of analog or electronic information relating to patient descriptions, including diagnosis, symptoms, exacerbations and treatment made by the treating physician, patient enrollment data, patient enrollment data, laboratory data, prescription drug data, insurance claims data, data from a diagnostic medial device (such as a heart monitor), etc.

### FIELD OF THE INVENTION

This invention is generally directed to a system and method of establishing a disease classification code for a patient from written or electronic patient medical records existing within a medical practice, a hospital, a clinic, or any other location maintaining medical records. More specifically, the system and method of the present invention utilizes actual patient chart data or medical records, which may be input into a hand-held device or directly into a computerized system, to establish confirmed (and accurate) industry standard disease classification codes (e.g., ICD-9 Codes from International Classification of Diseases, 9^{th} edition). Updated disease classification codes are then stored in a diagnosis repository database, and can ultimately be used to verify and compare diagnostic coding submitted by physicians when billing for services rendered to a patient. Concern about inaccurate coding is the greatest in physicians' offices, where appropriate diagnosis coding has not affected payment for services rendered, and concerns about proper disease coding is less rigorously practiced than in hospitals. Thus, although not limited to physicians' offices, the system and method of the present invention will perhaps prove most beneficial in increasing physician diagnosis coding specificity, which will then be used to adjust reimbursement from government agencies on a severity of illness basis, among other demographic aspects such as age, gender, geography, etc.

### BACKGROUND OF THE INVENTION

Managed care health insurance products or plans for populations of patients have long existed. In the past, the purpose of such plans was to demographically allocate the risk of medical costs over a large population. Government agencies, such as Medicare and Medicaid, would then reimburse such managed care insurance companies based on capitation payments to such plans, linked to “fee for service” expenditures by geographic area, with payments set at ninety-five percent (95%) of an enrollee's county's adjusted average per capita cost (AAPCC). The AAPCC payment methodology explains only about one percent (1%) of the variation in expenditures for Medicare beneficiaries, and does not pay more for sicker people. Thus, research has showed that the managed care program was increasing total Medicare Program expenditures, because its enrollees were healthier than fee for service enrollees, and the AAPCC did not account for this favorable selection. Also, additional funds were not directed to plans enrolling sicker beneficiaries, or to plans specializing in treating high-cost populations, such as beneficiaries with particular chronic diseases or high levels of functional impairment.

In 2000, the Centers for Medicare and Medicaid Services (“CMS”) implemented a new model as a health-based payment adjuster. This model estimates beneficiary health status (expected cost next year) from AAPCC-like demographics and the worst principal inpatient diagnosis (principal reason for inpatient stay) associated with any hospital admission. These severities of illness-based payments were introduced gradually, with only ten percent (10%) of total Medicare capitation payments adjusted by these factors in 2000. The other ninety percent (90%) of payments were still adjusted using a purely demographic (AAPCC-like) model. The risk-based model was intended as a transition, i.e., as a feasible way to implement risk adjustment based on the readily available, already audited inpatient diagnostic data. Relying on inpatient diagnoses is the model's major shortcoming, since only illnesses that result in hospital admissions are counted; Managed Care Organizations that reduce admissions (e.g., through good ambulatory care) can end up with apparently healthier patients and lower payments. Congress ultimately addressed these limitations by requiring the use of ambulatory diagnoses in Medicare risk adjustment, to be phased in from 2004 to 2007 at thirty (30), fifty (50), seventy-five (75), and one hundred (100) percent, respectively, of total payments.

Under Section 231 of the Medicare Modernization Act of 2003, Congress created a new type of Medicare Advantage coordinated care plan focused on individuals with special needs. As a result of this legislation, these types of plans are not intended to be constructed or operated as traditional Medicare contracting, discounting or “gate-keeping” health management organizations (HMOs). Rather, they are designed and operated as clinical programs requiring special expertise in community coordinated care with both physician and patient emphasis. “Special Needs Plans” (SNPs) were identified by Congress as special Medicare health plans that serve only 1) institutionalized; 2) dually eligible; and/or 3) individuals with severe or disabling chronic conditions.

In order to fully implement its new policy, the United States government has created certain SNPs that consist solely of patients falling under a particular Hierarchical Condition Category (“HCC”) such as diabetes, kidney failure, cardiac disease, etc. Obviously, there are different severities of such diseases. Thus, in order to ascertain an appropriate level of reimbursement to such SNPs and also to determine which plans are successfully treating chronically ill patients, the government typically provides reimbursement based on pre-established disease classification codes mapped to specific HCCs. One such widely established and accepted coding system is the International Classification of Diseases (“ICD”). Since the insurance company will be reimbursed, audited and evaluated based on the submission of data establishing the severity of particular illnesses within its population of enrollees, it is essential that a fully defined, reported and correct disease classification code be established for each enrollee or patient assigned to a health plan. Regrettably, since physicians are not obligated to report, for reimbursement purposes, a full description by ICD-9 nomenclature or diagnosis code, of the illnesses or diagnoses of patients, physician coding specificity is severely lacking. The lack of effective coding by a physician is particularly regrettable in the context of a coded-based reimbursement system since it is the physician that typically maintains the most reliable, up-to-date medical information about a patient's condition.

Physicians often neglect to update their coding, particularly among chronically ill patients. For instance, a doctor that has long been treating a patient with Type II diabetes might neglect to update the classification code of the patient should an amputation follow. The result will be reimbursement from the government in an amount less than the government would pay based on this new clinical complication from the diabetes. In the past, doctors and insurance plans did not need to update classification codes as accurately since reimbursement was based on broad demographic data as opposed to the newer technique of reimbursing medical plans based on the full individual disease burdened classification codes.

There exists in the prior art several software tools that would permit an insurance company to “mine” insurance claims data to determine anomalies or suspected deficiencies of disease classification codes within a population. For instance, if the average occurrence within a particular population of diabetes patients having limb amputations is four percent (4%), and a particular facility has a rate of only one percent (1%), the software tool will identify this anomaly or suspected deficiency and target that facility for disease classification coding adjustment. The problem with software applications that simply identify deviations beyond an accepted range is that they are based on empirical data—not actual patient conditions as reflected in the patient's medical record. Thus, much wasted effort could be expended targeting coding techniques at a particular facility wherein, in actuality, the facility simply has encountered a higher (and statistically inconsistent) number of diabetes patients with amputations.

It is therefore a primary object of the present invention to provide a new and improved system and method for improving physician coding specificity, accuracy and reliability by establishing a confirmed disease classification code based on actual patient data.

It is yet another object of the present invention to provide a new and improved system and method for improving physician coding specificity, accuracy and reliability by establishing a confirmed disease classification code wherein patient data is collected from a review of the patient's medical chart.

It is still a further object of the present invention to provide a new and improved system and method for improving physician coding specificity, accuracy and reliability by establishing a confirmed disease classification code wherein patient data is collected from a review of the patient's medical chart and is compared to the results of a medical chart abstraction tool.

It is an additional object of the present invention to provide a new and improved system and method for improving physician coding specificity, accuracy and reliability by establishing a confirmed disease classification code based on patient data from a patient's medical chart wherein the reviewer of such information correlates the results of a medical chart abstraction tool of such patient data by means of a hand-held electronic device.

It is still an additional object of the present invention to provide a new and improved system and method for improving physician coding specificity, accuracy and reliability by establishing a confirmed disease classification code wherein said confirmed disease classification code is stored in a diagnosis repository database.

It is yet a further object of the present invention to provide a new and improved system and method for improving physician coding specificity, accuracy and reliability by establishing a confirmed disease classification code wherein if said confirmed disease classification code is different than a pre-established or physician reported disease classification code for a particular patient, such information is forwarded to either the treating physician and/or any other agency requiring such information.

Other objects and advantages of the present invention will become apparent from the specification and the drawings.

### SUMMARY OF THE INVENTION

Briefly stated and in accordance with the preferred embodiment of the present invention, a system and method for determining an updated disease classification code for a patient within a managed care population is described consisting of (i) a patient condition processing unit for receiving a plurality of patient-related data, (ii) a diagnosis repository database coupled to the patient condition processing unit for storing a pre-established disease classification code for the patient, and (iii) a disease classification code application tool designed to convert medical chart data of the patient into an observed disease classification code for the patient wherein the observed disease classification code is forwarded to the patient condition processing unit and stored in a diagnosis repository database as the updated disease classification code. The updated disease classification code can then be forwarded to the treating physician, reimbursement agency, or any other agency requiring such data. The patient-related data can consist of analog or electronic information relating to patient descriptions, including diagnosis, symptoms, exacerbations, treatment made by the treating physician, patient enrollment data, laboratory data, prescription drug data, insurance claims data, data from a diagnostic medial device (such as a heart monitor), etc.

### BRIEF DESCRIPTION OF THE DRAWINGS

While the specification concludes with claims particularly pointing out and distinctly claiming the subject matter regarded as the invention herein, it is believed that the present invention will be more readily understood upon consideration of the following description, taken in conjunction with the accompanying drawings, wherein:

FIG. 1 is a schematic illustration of the system for determining an updated disease classification code for a patient within a managed care population in accordance with the present invention;

FIGS. 2A and 2B depict a schematic flow chart of a general overview of the operation of a disease classification tool utilized in conjunction with the present invention;

FIGS. 3A and 3B depict a schematic flow chart of the functional operation of a disease classification tool utilized in conjunction with the present invention; and

FIGS. 4A and 4B depict a schematic flow chart reflecting the method of submitting a confirmed disease classification code determined in accordance with the present invention.

### DESCRIPTION OF THE PREFERRED EMBODIMENT

The preferred embodiment of the system and method of the present invention will be described herein in connection with the establishment of a confirmed disease classification code in connection with a population of patients having diabetes. However, it will be readily noted that the invention is equally applicable to other diseases such as coronary artery disease (CAD), heart failure (HF), cerebrovascular disease (CVD), etc. Moreover, while the system and method of the present invention is described as ascertaining a confirmed disease classification code for one particular patient, it should be understood that numerous patients can be coded simultaneously. Moreover, while the following description of the preferred embodiment will be described with respect to establishing ICD-9 codes, it should be evident that any disease classification coding system can be utilized—whether internationally accepted or internally established.

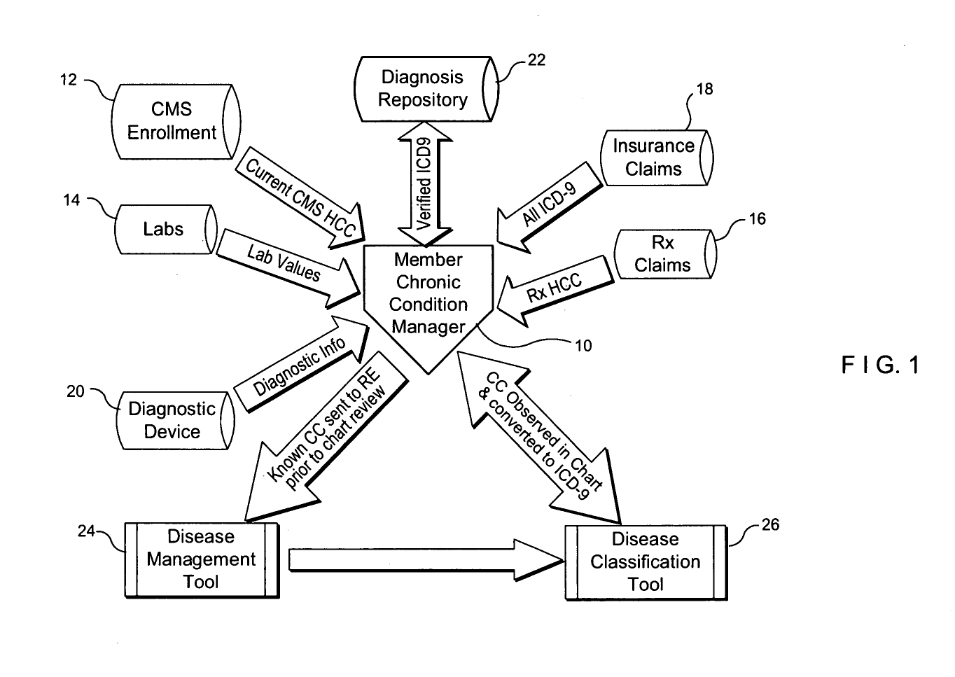

Referring to FIG. 1, a processing unit 10 (described as a member chronic condition manager), is shown receiving data from a CMS enrollment database 12, a laboratory database 14, a prescription drugs claims database 16, an insurance claims database 18, and a diagnostic device 20. CMS enrollment database 12 provides processing unit 10 with identification information and current CMS HCC information. Laboratory database 14 provides processing unit 10 with information concerning medical test results, etc. Prescriptions drugs claims database 16 provides processing unit 10 with prescription drug information for a particular patient. Insurance claims database 18 provides the claims experience for a particular patient, perhaps including the identification of the patient's currently used ICD-9 code. Diagnostic device 20 can be a heart monitor, etc. and can provide either real-time diagnostic data or stored diagnostic data for a particular patient to processing unit 10. Processing unit 10 would preferably receive information from databases 12, 14, 16, and 18, as well as from diagnostic device 20. However, it will be understood that any combination of such patient-related data can be utilized. Moreover, the described databases can be either combined or supplemented with yet additional patient-related data.

A diagnostic repository database 22 is also bi-directionally coupled to processing unit 10. Diagnosis repository database 22 stores ICD-9 codes for the population of patients within the patient population (e.g., within an SNP). Diagnostic repository database 22 can supply information to processing unit 10 or receive updated ICD-9 coding information from processing unit 10.

Processing unit 10 is also depicted as providing information to a chart abstraction tool 24. Chart abstraction tool 24 is a disease management tool such as the ohms/cad® disease management system provided by the assignee of the present invention, QMed, Inc. of Eatontown, N.J. Chart abstraction tool 24 is capable of interpreting patient data to provide recommended therapies. In the preferred embodiment of the present invention, chart abstraction tool 24 will be updated and run prior to initializing a disease classification tool 26. In other possible applications, disease classification tool 26 can be operated independent of disease management tool 24. Thus, processing unit 10 is shown to also be able to forward data directly to disease classification tool 26. In operation, personnel reviewing patient medical charts will operate disease management tool 24 and disease classification tool 26, preferably through a hand-held electronic device, to permit disease classification tool 26 to establish an updated and observed disease classification code (ICD-9 code) and provide it back to processing unit 10. Processing unit 10 will then store the updated and confirmed disease classification code (ICD-9 code) in diagnosis repository database 22. Chart abstraction tool 24 and disease classification tool 26 are typically software applications that can be accessed remotely through the Internet or installed within a hand-held electronic device.

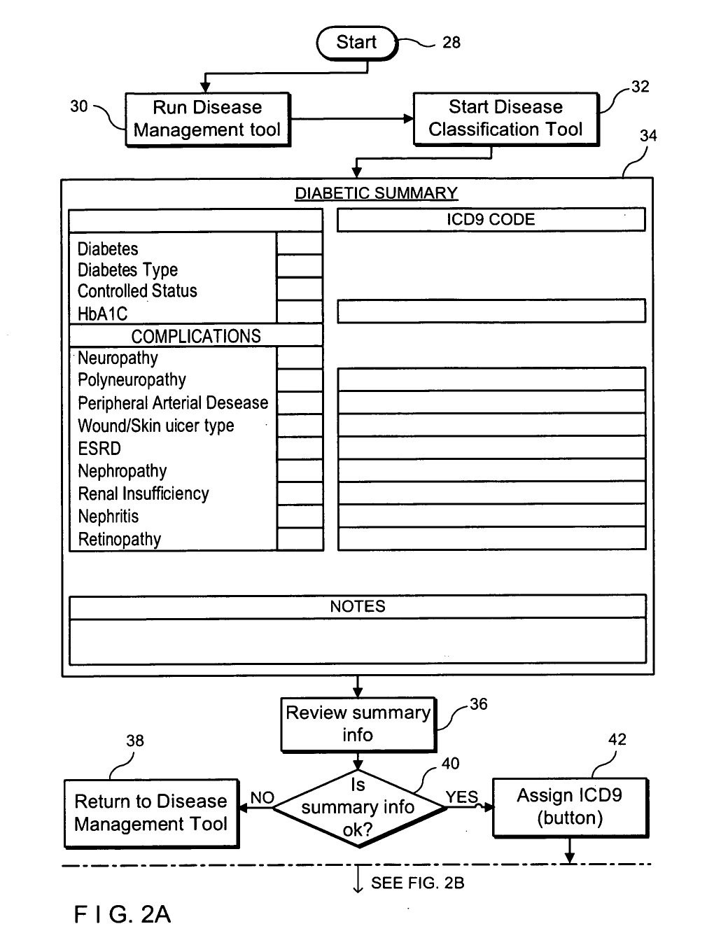

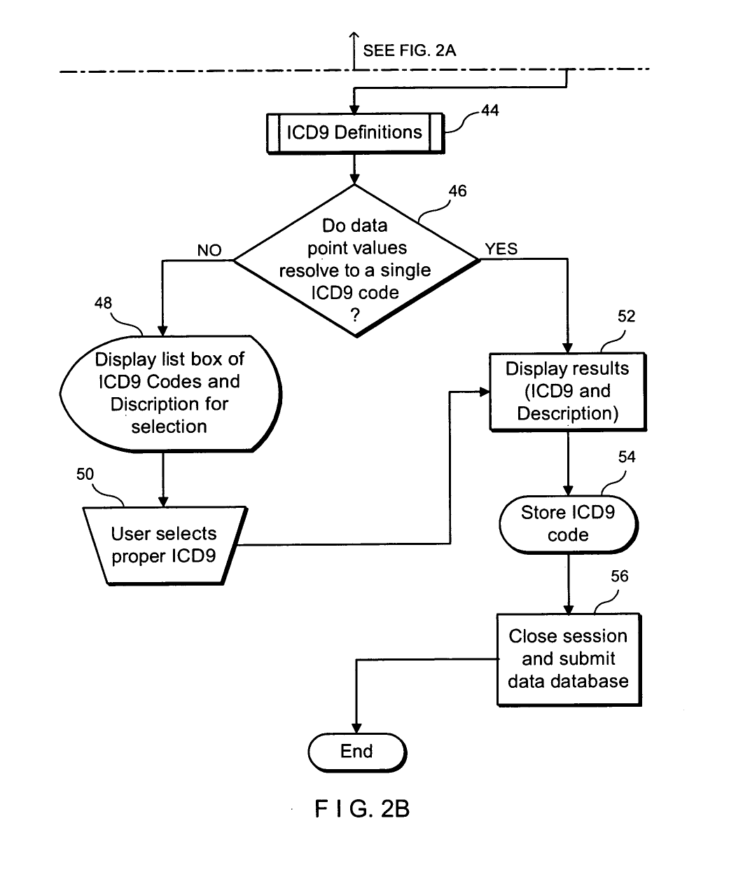

Referring next to FIG. 2, a flow chart describing a general overview of the system and method of the present invention is illustrated. It will be noted that the medical chart review as envisioned in connection with the system and method of the present invention can occur either at a remote facility (e.g., a doctor's office) or at a centralized location. Particularly in instances wherein the review is conducted remotely, the use of a hand-held electronic device is most advantageous.

After initializing the program (box 28), the user will run disease management tool 24 (box 30). After completing the data entry into disease management tool 24, a validation algorithm is applied to each data point. At that stage, disease classification tool 26 will be initialized (box 32). A menu-driven screen 34 is shown and can be fully displayed on a hand-held electronic device carried by the user. The hand-held tool is not depicted since it can take any form of personal hand-held device. Various types of patient data obtained from the patient's medical chart can be analyzed and shown on screen 34.

The user will next review the summary information (box 36) and determine if the summary information is acceptable (box 38). If the summary information is unacceptable, the user will return to implementing disease management tool 24, as depicted in box 40. Alternatively, if the user determines that the summary information is acceptable (at box 38), a request would be made to launch the process to assign an ICD-9 code (box 42). The assignment of an ICD-9 code is conducted by comparing patient data (as depicted by particular data point values) to a stored set of ICD-9 definitions (box 44). If the observed data point values do not result in the assignment of a single ICD-9 code, a display list of ICD-9 codes and descriptions of the same are shown along with associated data points (box 48). At this point, the reviewer/user will manually select a proper ICD-9 code based on review of the data points from the patient's medical chart (box 50). At that point, the determined ICD-9 code and description is displayed (box 52), the selected ICD-9 code is stored (box 54), disease classification tool 26 is terminated, and the modified code is submitted to diagnosis repository database 22 (box 56). If, at box 46, a single ICD-9 code is established, the tool can automatically display the determined ICD-9 code and description (box 52), store the appropriate ICD-9 code (box 54), terminate disease classification tool 26, and submit the confirmed ICD-9 code to diagnosis repository database 22 (box 56). Disease classification tool 26 can also be programmed to require user confirmation even in instances where a single ICD-9 code is determined.

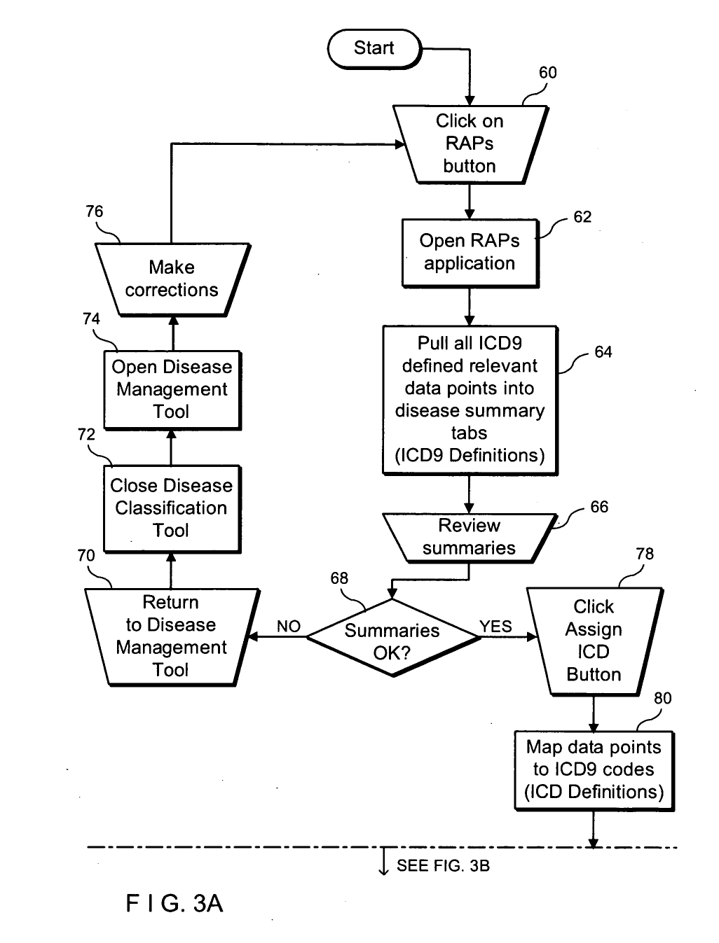

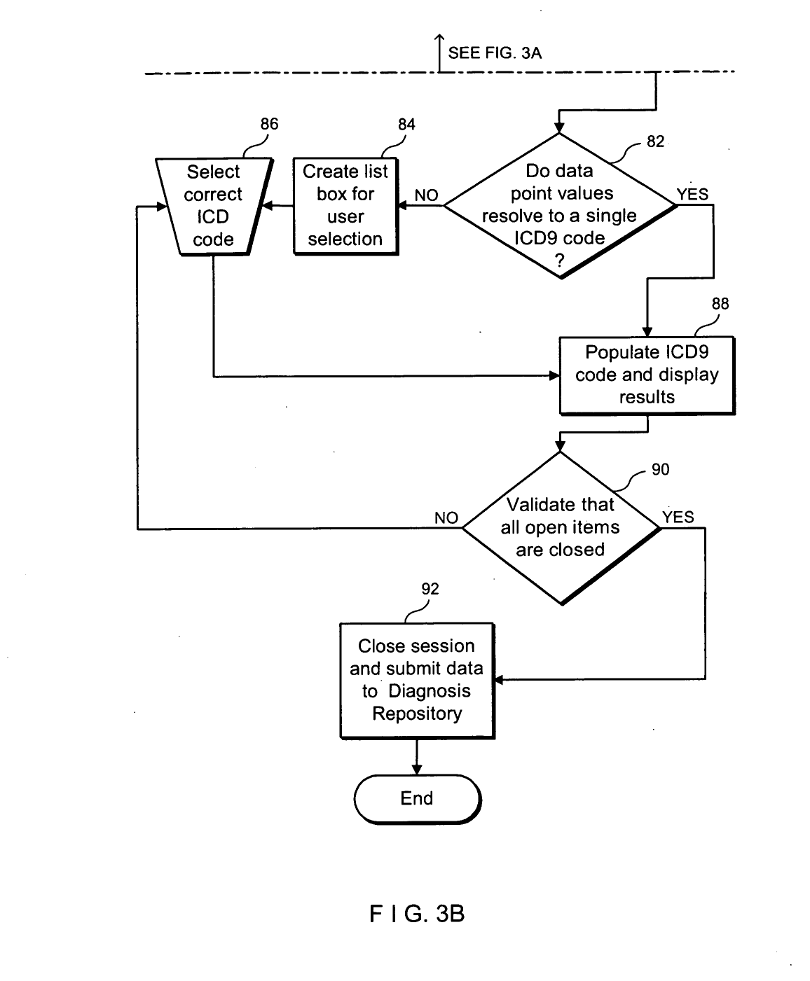

A more detailed functional analysis of disease classification tool 26 is depicted in FIG. 3. After the reviewer has run disease management tool 24, disease classification tool 26 is initialized (box 60). At this point, a visual display is made available (box 62) and all data points associated with relevant ICD-9 codes from the chart review are displayed (box 64). A summary report will be generated that can be reviewed by the user (box 66). If the summary is deemed non-acceptable by the user (box 68), the user will return to disease management tool 24 (at box 70), close disease classification tool 26 (at box 72), manually open disease management tool 24 (box 74) and make all necessary corrections (box 76). If, either initially or after making corrections through disease management tool 24, the summaries are deemed acceptable (box 68), the user will launch the process to assign a disease classification code (box 78), which will map all data points to ICD-9 codes (box 80) (the assignment of an ICD-9 code is conducted by comparing patient data, as depicted by particular data point values, to a stored set of ICD-9 definitions), and will then determine whether a single ICD-9 code has been derived (box 82). If no single ICD-9 code has been derived, a list box comprising possible ICD-9 codes and their definitions will be displayed (box 84), and a user will select a correct ICD-9 code (box 86) based on observed chart information. Once a user selects the correct ICD-9 code (at box 86), the selected code will be displayed (box 88). If, at decision box 82, a single ICD-9 code had been established that confirmed disease classification code could be displayed (box 88). In either case, the system will then make certain that all items are resolved (box 90). If all open items were indeed resolved, the session would be terminated and the results would be submitted to the diagnostic repository database 22 (box 92). If open items were not yet resolved, the user would once again be requested to select an appropriate ICD-9 code (box 86).

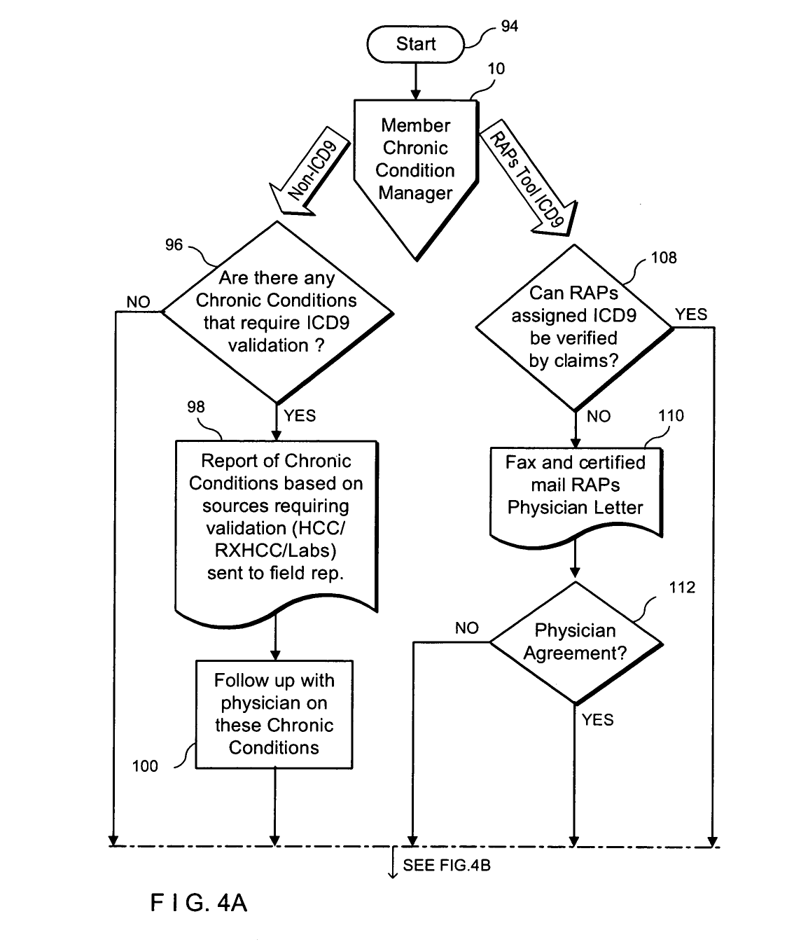

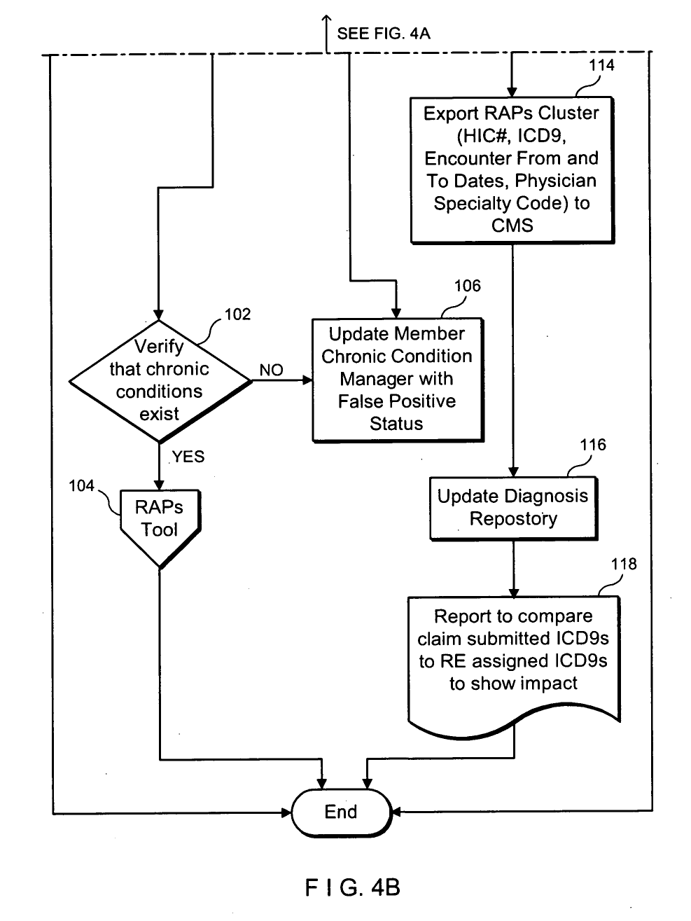

Once data-confirmed ICD-9 codes are reconciled against all known medical conditions for a patient and found not to be previously submitted, it is essential that the updated coding information be forwarded to the treating physician, reimbursement agency and/or any other entity requiring such data. Such data exchange can occur periodically (i.e., weekly, monthly, quarterly, etc.) or on demand. One example of this data exchange and reconciliation is depicted in flow chart format in FIG. 4. In FIG. 4, the term RAPS is an acronym for Risk Adjustment Processing Systems, which is dependent on physician coding specificity for accuracy and appropriate reimbursement, and is indicative of the results obtained from disease classification tool 26. First, the reconciliation process is initiated (box 94). Next, processing unit 10 determines if an ICD-9 code was established by running disease classification tool 26. If no ICD-9 code is established, the system will determine if any chronic conditions exist that require ICD-9 validation (box 96). If the answer at decision box 96 is no, the program is terminated. If chronic conditions do exist that require ICD-9 validation, based on patient data, a report is sent to a field representative (user), as reflected by box 98. The user will then consult with the treating physician (box 100) to determine whether the chronic conditions actually exist (decision box 102). If the chronic conditions do exist, disease classification tool 26 will be run (box 104). Conversely, if the chronic conditions do not exist, the processing unit will be updated to indicate a false positive status (box 106) before the program is terminated.

If, after initialization, processing unit 10 determines that an ICD-9 code does exist, either a program or a user can attempt to verify the ICD-9 code by comparing it to prior claims data (box 108) from the specified time frame. It will be noted by those skilled in the art that while the assigned ICD-9 code was verified in box 108, by comparison to claims data, the confirmation can also take place by comparing the assigned ICD-9 code to prescription drug data, data from a diagnostic device, etc. If the ICD-9 code is confirmed, the program is terminated. If the ICD-9 is non-analogous to the claims data, a report may be generated and forwarded to the physician (box 110) so that the physician can confirm or dispute the non-analogous results (box 112). If the physician determines that the newly assigned ICD-9 code is incorrect, processing unit 10 will be updated to indicate a false positive result (box 106) and the program will be terminated. Alternatively, if the physician agrees with the newly assigned and observed ICD-9 code, the modified ICD-9 code and related information will be forwarded to CMS or any the reimbursement agency and/or any other agency requiring such data (box 114) and diagnosis repository database 22 will be updated (box 116). Finally, a report may be generated to compare updated ICD-9 code(s) to the previously assigned ICD-9 code(s) to show potential impact (box 118).

It is unquestionably more likely that the newly observed ICD-9 code will be reflective of a more serious condition than a lesser condition since it is more likely that a condition/complication was overlooked as opposed to a condition/complication being mistakenly entered.

It will be apparent from the foregoing description that the present invention utilizes a novel system and method that permits the establishment and/or updating of disease classification codes. Many variations of the preferred embodiment are clearly envisioned. For instance, any number of servers can be interconnected to implement the present invention. Moreover, although the preferred embodiment was described in conjunction with a patient population having a single disease, numerous diseases can be considered within a single population.

While there has been shown and described what is presently considered to be the preferred embodiment of this invention, it will be obvious to those skilled in the art that various changes and modifications may be made without departing from the broader aspects of this invention. It is, therefore, aimed in the appended claims to cover all such changes and modifications as fall within the true scope and spirit of the invention.

We claim:

1. A method of establishing a disease classification code for a patient within a managed care population comprising the steps of:
reviewing patient data;
implementing a menu-driven disease classification tool to input said patient data;
comparing said patient data to a stored plurality of defined disease classification codes;
assigning a confirmed disease classification code to said patient based on said comparing step; and
storing said confirmed disease classification code in a diagnosis repository database.

2. The method of claim 1 further comprising the steps of:
comparing said confirmed disease classification code to a stored disease classification code in said diagnosis repository database; and
generating a modification report to identify any differences between said confirmed disease classification code and said stored disease classification code.

3. The method of claim 2 further comprising the step of:
forwarding said modification report to a treating physician's office of said patient.

4. The method of claim 2 further comprising the step of:
forwarding said modification report to a reimbursement agency.

5. The method of claim 1 wherein said step of reviewing patient data is performed by a user reviewing a patient medical chart.

6. A method of establishing a disease classification code for a patient within a managed care population comprising the steps of:
reviewing patient data;
implementing a menu-driven disease classification tool to input said patient data;
comparing said patient data to a stored plurality of defined disease classification codes;
determining whether said comparing step results in a determination of a single disease classification code or a plurality of possible disease classification codes;
assigning an automatically confirmed disease classification code to said patient when said determining step results in said single disease classification code;
displaying said plurality of possible disease classification codes and assigning a user-selected confirmed disease classification code when said determining step results in said plurality of a possible disease classification codes; and
storing said confirmed disease classification code in a diagnosis repository database.

7. The method of claim 6 further comprising the steps of:
comparing said confirmed disease classification code to a stored disease classification code in a said diagnosis repository database; and
generating a modification report to identify any differences between said confirmed disease classification code and said stored disease classification code.

8. The method of claim 6 further comprising the step of:
forwarding said modification report to a treating physician's office of said patient.

9. The method of claim 6 further comprising the step of:
forwarding said modification report to a reimbursement agency.

10. The method of claim 6 wherein said step of reviewing patient data is performed by a user reviewing a patient medical chart.

11. A method of utilizing personnel to operate and control both a disease management tool and a disease classification tool for a patient within a managed care population comprising the steps of:
utilizing said disease management tool to obtain a disease management report;
deploying said disease classification tool and inputting patient disease classification data;
generating a summary report from said disease classification tool;
evaluating said summary report from said disease management report;
modifying disease management data in a said disease management tool when said evaluating step depicts non-analogous results;
comparing said patient disease classification data to a stored plurality of defined disease classification codes when said evaluating step depicts analogous results;
assigning a confirmed disease classification code to said patient based on said comparing step; and
storing said confirmed disease classification code in a diagnosis repository database.

12. The method of claim 10 further comprising the steps of:
comparing said confirmed disease classification code to a stored disease classification code in said diagnosis repository database; and
generating a modification report to identify any differences between said confirmed disease classification code and said stored disease classification code.

13. The method of claim 12 further comprising the step of:
forwarding said modification report to a treating physicians office of said patient.

14. The method of claim 12 further comprising the step of:
forwarding said modification report to a reimbursement agency.

15. A method of utilizing personnel to operate and control both a disease management tool and a disease classification tool for a patient within a managed care population comprising the steps of:
utilizing said disease management tool to obtain a disease management report;
deploying said disease classification tool and inputting patient disease classification data;
generating a summary report from said disease classification tool;
evaluating said summary report and said disease management report modifying disease management data in said disease management tool when said evaluation step depicts non-analogous data;
comparing said patient disease classification data to a stored plurality of defined disease classification codes when said evaluation step depicts analogous results;
determining whether said comparing step results in a determination of a single disease classification code or a plurality of possible disease classification codes;
assigning an automatically confirmed disease classification code to said patient when said determining step results in said single disease classification code;
displaying said plurality of possible disease classification codes and assigning a user-selected confirmed disease classification code when said determining step results in said plurality of possible disease classification codes; and
storing said confirmed disease classification code in a diagnosis repository database.

16. The method of claim 15 further comprising the steps of:
comparing said confirmed database classification code to a stored disease classification code in a said diagnosis repository database; and
generating a modification report to identify any differences between said confirmed disease classification code and said stored disease classification code.

17. The method of claim 16 further comprising the step of:
forwarding said modification report to a treating physicians office of said patient.

18. The method of claim 16 further comprising the step of:
forwarding said modification report to a reimbursement agency.

19. A system for determining an updated disease classification code for a patient within a managed care population comprising:
a patient condition processing unit for receiving a plurality of patient-related data;
a diagnosis repository database coupled to said patient condition processing unit for storing a pre-established disease classification code for said patient; and
a disease classification code application tool designed to convert medical chart data of said patient into an observed disease classification code for said patient wherein said observed disease classification code is forwarded to said patient condition processing unit and stored in a diagnosis repository database as said updated disease classification code.

20. The system of claim 19 wherein said patient-related data includes patient enrollment data within said managed care population.

21. The system of claim 19 wherein said patient-related data includes laboratory data.

22. The system of claim 19 wherein said patient-related data includes prescription drug data.

23. The system of claim 19 wherein said patient-related data includes insurance claims data.

24. The system of claim 19 wherein said patient-related data includes diagnostic data from a diagnostic medical device.

25. The system of claim 19 wherein said disease classification code application tool is contained in a hand-held electronic device.

26. The system of claim 25 wherein said disease classification code application tool is menu-driven.

27. The system of claim 19 wherein said medical chart data is obtained by operating a chart abstraction tool.

28. A method of updating a preestablished disease classification code for a patient within a managed care population comprising the steps of:
storing a pre-established disease classification code for said patient;
reviewing medical chart data of said patient;
utilizing a disease classification code application tool to convert said medical chart data into an observed disease classification code; and
replacing said pre-established disease classification code with said observed disease classification code.

29. The method of claim 28 further comprising the steps of:
comparing said observed disease classification code to said pre-established disease classification code; and
generating a modification report to identify any differences between said pre-established disease classification code and said observed disease classification code.

30. The method of claims 29 further comprising the step of:
forwarding said modification report to a treating physicians office of said patient.

31. The method of claim 29 further comprising the step of:
forwarding said modification report to a reimbursement agency.
